(19) United States
(12) Patent Application Publication
(10) Pub. No.: US 2010/0179175 A1
(43) Pub. Date: Jul. 15, 2010

(54) USE OF PKC INHIBITORS IN OCULAR DISEASES

(76) Inventors: Jeremy M. Sivak, Rosindale, MA (US); Jürgen Wagner, Bottmingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/377,165

(22) PCT Filed: Aug. 21, 2007
(86) PCT No.: PCT/US07/76361

§ 371 Date: Feb. 11, 2009

Related U.S. Application Data
(60) Provisional application No. 60/823,298, filed on Aug. 23, 2006.
(60) Provisional application No. 60/914,899, filed on Apr. 30, 2007.

(51) Int. Cl. A61K 31/517; A61K 31/438; A61P 27/02

(52) U.S. Cl. 514/266.2; 514/278

(57) ABSTRACT

The present invention pertains to the use of a PKC inhibitor in the treatment of an ocular disorder.

The present invention relates to the use of a PKC inhibitor in the treatment or prevention of ocular diseases and disorders, in particular involving inflammation and/or neovascularization, such as macular degeneration (AMD), uveitis, diabetic retinopathy or diabetic macular edema.

Macular degeneration is an incurable eye disease that leads to irreversible loss of central vision. It is the most common cause of blindness in people aged 55 and older. As people age, their chances for developing eye diseases, and in particular AMD, increase dramatically.

Age-related macular degeneration (ARMD) is the most common form of macular degeneration. It is also known as age-related maculopathy (ARM), aged macular degeneration, and senile macular degeneration.

Uveitis is a condition of ocular inflammation, in particular of the uveal tract. This includes inflammation of the iris, ciliary body, and choroid. Depending on the side of inflammation, it can also be described as anterior uveitis, intermediate uveitis, posterior uveitis, or pan-uveitis.

In spite of numerous treatment options for treating or preventing these diseases and disorders, disease continues to progress and there remains a need for effective and safe treatment.

The present invention provides the use of a PKC inhibitor, in particular an indolylmaleimide derivative in preventing or treating or delaying ocular diseases and disorders involving inflammation and/or neovascularization, wherein the indolylmaleimide derivative is a compound of formula (I)

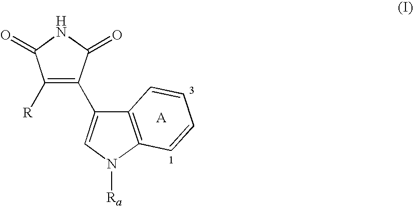

wherein
Rₐ is H; C₁₋₄alkyl; or C₁₋₄alkyl substituted by OH, NH₂, NHC₁₋₄alkyl or N(di-C₁₋₄alkyl)₂; and
R is a radical of formula (a) or (b)

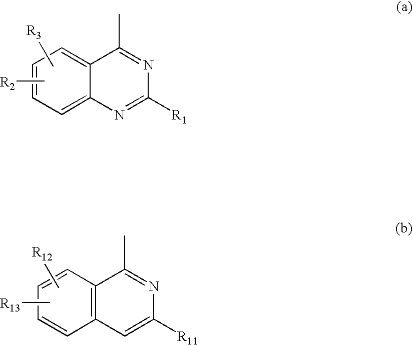

wherein
each of R₁ and R₁₁ is a heterocyclic residue; NR₄R₅ wherein R₄ and R₅ form together with the nitrogen atom to which they are bound a heterocyclic residue;
each of R₂, R₃, R₁₂ and R₁₃, independently, is H, halogen, C₁₋₄alkyl, CF₃, OH, SH, NH₂, C₁₋₄alkoxy, C₁₋₄alkylthio, NHC₁₋₄alkyl, N(di-C₁₄alkyl)₂ or CN; and
ring A is optionally substituted,
or a pharmaceutically acceptable salt thereof.

In formula (I), any alkyl or alkyl moiety in e.g. alkoxy may be linear or branched. Halogen may be F, Cl, Br or I, preferably F or Cl. Any aryl may be phenyl or naphthyl, preferably phenyl.

By heterocyclic residue as R₁, or R₁₁, or formed by NR₄R₅, is meant a three to eight, preferably five to eight, membered saturated, unsaturated or aromatic heterocyclic ring comprising 1 or 2 heteroatoms, preferably selected from N, O and S, and optionally substituted.

Suitable examples of heterocyclic residue as R₁, R₁₁ or formed by NR₄R₅ include e.g. pyridyl, e.g. 3- or 4-pyridyl, piperidyl, e.g. piperidin-1-yl, 3- or 4-piperidyl, homopiperidyl, piperazinyl, e.g. 1-piperazinyl, homopiperazinyl, morpholin-4-yl, imidazolyl, imidazolidinyl, pyrrolyl or pyrrolidinyl, optionally substituted, e.g. mono- or polysubstituted. When the heterocyclic residue is substituted, this may be on one or more ring carbon atoms and/or on a ring nitrogen atom when present. Examples of a substituent on a ring carbon atom include e.g. C₁₋₄alkyl e.g. CH₃;

C₃₋₆cycloalkyl e.g. cyclopropyl, optionally further substituted by C₁₋₄alkyl;

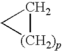

wherein p is 1,2 or 3, preferably 1; CF₃; halogen; OH; NH₂; —CH₂—NH₂; —CH₂—OH; piperidin-1-yl; or pyrrolidinyl. Examples of a substituent on a ring nitrogen atom are e.g. C₁₋₆alkyl; acyl, e.g. R′ₓ—CO wherein R′ₓ is H, C₁₋₆alkyl or phenyl optionally substituted by C₁₋₄alkyl, C₁₋₄alkoxy or amino, e.g formyl; C₃₋₆cycloalkyl; C₃₋₆cycloalkyl-C₁₋₄alkyl; phenyl; phenyl-C₁₋₄alkyl e.g. benzyl; a heterocyclic residue, e.g. as disclosed above, e.g. an aromatic heterocyclic residue comprising 1 or 2 nitrogen atoms; or a residue of formula α

—R₂₁—Y′ (α)

wherein R₂₁ is C₁₋₄alkylene or C₂₋₄alkylene interrupted by O and Y′ is OH, NH₂, NE(C₁₋₄alkyl) or N(C₁₋₄alkyl)₂.

In formula (I) C₂₋₄alkylene interrupted by O may be e.g. —CH₂—CH₂—O—CH₂—CH₂—.

In formula (I), when the substituent on a cyclic nitrogen is a heterocyclic residue, it may be a five or six membered saturated, unsaturated or aromatic heterocyclic ring comprising 1 or 2 heteroatoms, preferably selected from N, O and S. Examples include e.g. 3- or 4-pyridyl, piperidyl, e.g. piperidin-1-yl, 3- or 4-piperidyl, homopiperidyl, piperazinyl, homopiperazinyl, pyrimidinyl, morpholin-4-yl, imidazolyl, imidazolidinyl, pyrrolyl or pyrrolidinyl,

In formula (I), when Rₐ is substituted C₁₋₄alkyl, the substituent is preferably on the terminal carbon atom.

When ring A is substituted, it may be mono- or polysubstituted, preferably monosubstituted, the substituent(s) being selected from the group consisting of e.g. halogen, OH, C₁₋₄alkoxy, e.g. OCH₃, C₁₋₄alkyl, e.g. CH₃, NO₂, CF₃, NH₂, NHC₁₋₄alkyl, N(di-C₁₋₄alkyl)₂ and CN. For example, ring A may be a residue of formula

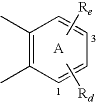

wherein
R_{d} is H; C₁₋₄alkyl; or halogen; and
Rₑ is OH; NO₂; NH₂; NHC₁₋₄alkyl; or N(di-C₁₋₄alkyl)₂.

Preferably R_{d} is in position 1; preferably Rₑ is in position 3.

When R_{c} has a CH₂ replaced by CRₓR_{y}, it is preferably the CH₂ bearing Y.

Examples of heterocyclic residue as R₁, R₁₁, or formed by NR₄R₅ include e.g. a residue of formula (γ)

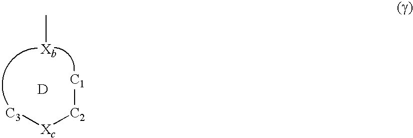

wherein
the ring D is a 5, 6 or 7 membered saturated, unsaturated or aromatic ring;
X_{b} is —N—, —C═ or —CH—;
Xₓ is —N═, —NR_{f}—, —CR═ or —CHR_{f}′— wherein R_{f} is a substituent as indicated above for a ring nitrogen atom, and R_{f}′ is a substituent as indicated above for a ring carbon atom;
the bond between C₁ and C₂ is either saturated or unsaturated;
each of C₁ and C₂, independently, is a carbon atom which is optionally substituted by one or two substituents selected among those indicated above for a ring carbon atom; and
the line between C₃ and X_{b} and between C₁ and X_{b}, respectively, represents the number of carbon atoms as required to obtain a 5, 6 or 7 membered ring D.

A preferred residue of formula (γ) is one wherein the ring D forms a 1,4-piperazinyl ring optionally C- and/or N-substituted as indicated.

Representative examples of a residue of formula (γ) are e.g. 3- or 4-pyridyl; piperidin-1-yl; 1-N—(C₁₋₄alkyl)- or -(ω-hydroxy-C₁₋₄alkyl)-3-piperidyl; morpholin-4-yl; imidazolyl; pyrrolidinyl; 1-piperazinyl; 2-C₁₋₄alkyl- or —C₃₋₆cycloalkyl-1-piperazinyl; 3-C₁₋₄alkyl- or —C₃₋₆cycloalkyl-1-piperazinyl; 2,2- or 3,5- or 2,5- or 2,6-di(C₁₋₄alkyl)-1-piperazinyl; 3,4,5-tri-(C₁₋₄alkyl)-1-piperazinyl; 4-N—(C₁₋₄alkyl)- or -(ω-hydroxy-C₁₋₄alkyl)- or -(ω-dimethylamino-C₁₋₄alkyl)-1-piperazinyl; 4-N-pyridin-4-yl-1-piperazinyl; 4-N-phenyl- or —C₃₋₆cycloalkyl-1-piperazinyl; 4-N—(C₁₋₄alkyl)- or -(ω-hydroxy-C₁₋₄alkyl)-3-C₁₋₄alkyl- or -3,3-di(C₁₋₄alkyl)-1-piperazinyl; 4-N-(1-C₁₋₄alkyl-C₃₋₆cycloalkyl)-1-piperazinyl; 4-N-formyl-1-piperazinyl; 4-N-pyrimidin-2-yl-1-piperazinyl; 4,7-diaza-spiro[2.5]oct-7-yl or 4-N—C₁₋₄alkyl-1-homopiperazinyl.

The compounds of formula (I) may exist in free form or in salt form, e.g. addition salts with e.g. organic or inorganic acids, for example, hydrochloric acid, acetic acid, when R₁ or R₁₁ and/or R₂, R₃, R₁₂ or R₁₃ comprises an optionally substituted amino group or a heterocyclic residue which can form acid addition salts.

It will be appreciated that the compounds of formula (I) may exist in the form of optical isomers, racemates or diastereoisomers. For example, a ring carbon atom bearing a substituent in the heterocyclic residue as R₁, R₁₁ or formed by NR₄R₅ is asymmetric and may have the D- or L-configuration. It is to be understood that the present invention embraces all enantiomers and their mixtures. Similar considerations apply in relation to starting materials exhibiting asymetric carbon atoms as mentioned.

In the compounds of formula (I), the following significances are preferred individually or in any sub-combination:
1. Rₐ is H or CH₃;
2. R_{b} is H;
3. Ring A is unsubstituted; or is substituted by methyl in position 7;
4. Preferred heterocyclic residue as formed by NR₄R₅ is e.g. piperazin-1-yl optionally N-substituted, e.g. by C₁₋₄alkyl, ω-hydroxy-C₁₋₄alkyl, ω-dimethylamino-C₁₋₄alkyl, C₅₋₆cycloalkyl, C₁₋₄alkyl-C₅₋₆cycloalkyl, an aromatic heterocyclic residue comprising 1 or 2 nitrogen atoms, e.g. pyridyl or pyrimidin-2-yl or 4,7-diaza-spiro[2.5]oct-7-yl; or a residue of formula β as defined above and/or optionally C-substituted, e.g. by CH₃ e.g. in positions 2, and/or 3 and/or 5 and/or 6 and/or 2,2 or 3,3 or by

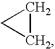

e.g. in position 2 or 3; piperidin-1-yl optionally C-substituted, e.g. in position 4, by NH₂, —CH₂—NH₂ or piperidin-1-yl, or in position 3, e.g. by OH or NH₂; or pyrrolidinyl optionally C-substituted in position 3 by OH or NH₂;
5. Each of R₁ and R₁₁, independently, is 1-N-methyl-piperidin-4-yl; 4-methyl-piperazin-1-yl; 4-methyl-1-homopiperazinyl; 4-(2-hydroxyethyl)-piperazin-1-yl; or —X′—C_{1,2 or 3}-alkylene-NR₇R₈ wherein X′ is a direct bond, O or NH;
6. In the residue of formula (a) either each of R₂ and R₃ is H or one of R₂ and R₃ is H and the other is F, Cl, CH₃, OCH₃ or CF₃;
7. In the residue of formula (a) either each of R₁ and R₂ is H or one of R₁ and R₂ is H and the other is F, Cl, CH₃, OCH₃ or CF₃; preferably R₂ is H and R₁ is in position 5, 6, 7 or 8, preferably in position 6;
8. In the residue of formula (b) each of R₁₂ and R₁₃ is H; or one of R₁₂ and R₁₃ is H and the other is F, Cl, CH₃, OCH₃ or CF₃; preferably R₁₃ is H and R₁₂ is in position 7;
9. In the residue of formula (b), each of R₁₂ and R₁₃ is H; R₁₁ is 4,7-diaza-spiro[2.5]oct-7 yl; or piperazin-1-yl substituted in position 3 by methyl or ethyl and optionally in position 4 by methyl.

The compounds of formula (I) are known and may be prepared as disclosed in the art, e.g. as described in U.S. Pat. No. 6,645,970, EP1490355A1, which are incorporated herein by reference. They may be prepared as disclosed or by analogy to the procedures described in these references.

Preferred compounds of formula (I) are 3-(1.H.-indol-3-yl)-4-[2-(4-methyl-piperazin-1-yl)-quinazolin-4-yl]-pyrrole-2,5-dione (referred to hereinafter as Compound A), 3-(1.H.-indol-3-yl)-4-[2-(piperazin-1-yl)-quinazolin-4-yl]-pyrrole-2,5-dione (referred to hereinafter as Compound B), 3-[3-(4,7-Diaza-spiro[2.5]oct-7-yl)-isoquinolin-1-yl]-4-(7-methyl-1H-indol-3-yl)-pyrrole-2,5-dione (Compound C), in free form or in a pharmaceutically acceptable salt form, e.g. the acetate salt of 3-(1.H.-indol-3-yl)-4-[2-(4-methyl-piperazin-1-yl)-quinazolin-4-yl]-pyrrole-2,5-dione, or 3-[3-(4,7-Diaza-spiro[2.5]oct-7-yl)-isoquinolin-1-yl]-4-(7-methyl-1H-indol-3-yl)-pyrrole-2,5-dione.

Other PKC inhibitors to be used in accordance of the invention are compounds of formula IIa

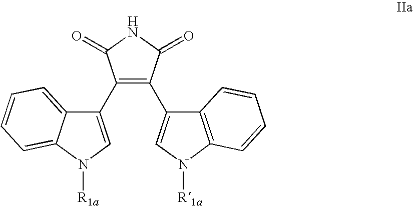

wherein
R₁ₐ is

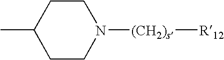

wherein either s′ is 0 and R′₁₂ is hydrogen or C₁₋₄alkyl; or s′ is 1 and R′₁₂ is pyridyl, preferably 2-pyridyl, and
R′₁ₐ is hydrogen or C₁₋₄alkyl,
or a pharmaceutically acceptable salt thereof.

The compounds of formula IIa may exist in form of hydrate or solvate.

Even more preferred are 3-(1-methyl-1H-indol-3-yl)-4-[1-{(1-pyridin-2-ylmethyl)-piperidin-4-yl}-1H-indol-3-yl]-pyrrole-2,5-dione (Compound D), or 3-(1-methyl-1H-indol-3-yl)-4-[1-(piperidin-4-yl)-1H-indol-3-yl]-pyrrole-2,5-dione (Compound E), or a pharmaceutically acceptable salt, hydrate or solvate thereof.

The compounds of formula IIa may be synthesized as known in the art, e.g. as described in U.S. Pat. No 5,545,636.

In a series of further specific or alternative embodiments, the present invention also provides:
1. A method for treating, preventing or delaying ophthalmic diseases and disorders in particular involving inflammatory and/or neovascular events, or delaying their progression, as described hereinbelow, said method comprising administering to an affected subject a therapeutically effective amount of a PKC inhibitor, e.g. a compound of formula I or a compound of formula IIa.

As herein defined, “neovascularization”, also called “neovascular events” include, but is not limited to retinal neovascularization, corneal neovascularization and choroidal neovascularization.

“Ocular diseases or disorders involving inflammatory and/or neovascular events” as defined in this application comprises, but is not limited to macular degeneration (AMD), diabetic ocular diseases or disorders, uveitis, optic neuritis, ocular edema, ocular angiogenesis, ischemic retinopathy, anterior ischemic optic neuropathy, optic neuropathy and neuritis, macular edema, cystoid macular edema (CME), retinal disease or disorder, such as retinal detachment, retinitis pigmentosa (RP), Stargart's disease, Best's vitelliform retinal degeneration, Leber's congenital amaurosis and other hereditary retinal degenerations, Sorsby's fundus dystrophy, pathologic myopia, retinopathy of prematurity (ROP), Leber's hereditary optic neuropathy, corneal transplantation or refractive corneal surgery, keratoconjunctivitis, or dry eye.

As herein defined, “AMD” includes but is not limited to age-related macular degeneration (ARAMD). ARMD includes its dry forms (dry ARMD) and wet forms (wet ARMD).

“Diabetic ocular diseases or disorders” as defined in this application comprises, but is not limited to, diabetic retinopathy (DR), diabetic macular edema (DME), proliferative diabetic retinopathy (PDR) and uveitis.

“Uveitis” as defined in the application comprises, but is not limited to anterior uveitis, intermediate uveitis, posterior uveitis, and panuveitis.

In another aspect the present invention provides:
2. A PKC inhibitor, e.g. a compound of formula (I) or (IIa), preferably Compound A, B, C, D or E, or a pharmaceutically acceptable salt thereof, for use in a method as defined under 1 above;
3. A PKC inhibitor, e.g. a compound of formula (I) or (IIa), preferably Compound A, B, C, D or E, or a pharmaceutically acceptable salt thereof, for use in the preparation of a pharmaceutical composition for use in a method as defined under 1 above;
4. Use of a PKC inhibitor, e.g. a compound of formula (I) or (IIa), preferably Compound A, B, C, D or E, or a pharmaceutically acceptable salt thereof, in the preparation of a medicament for treating, preventing or delaying ophthalmic diseases and disorders in particular involving inflammatory and/or neovascular events, or delaying their progression, in particular age-related macular degeneration, retinal disease or disorder or diabetic ocular diseases or disorders as hereinabove defined.
5. A pharmaceutical composition for use in a method as defined under 1 above comprising a PKC inhibitor, e.g. a compound of formula (I) or (IIa), preferably Compound A, B, C, D or E, or a pharmaceutically acceptable salt thereof, together with one or more pharmaceutically acceptable diluents or carriers therefor.

The compounds of formula (I) may be administered in free form or in pharmaceutically acceptable salt form e.g. as indicated above. Such salts may be prepared in conventional manner and exhibit the same order of activity as the free compounds.

The compounds of formula (IIa) may be administered in free form or in form of hydrate, solvate or salt, e.g. in a pharmaceutically acceptable salt form. Such hydrates, solvates and salts may be prepared in conventional manner and exhibit the same order of activity as the free compounds.

Utility of the PKC inhibitor, e.g. in the treatment of ophthalmic diseases and disorders involving inflammatory or neovascular event, as hereinabove specified, may be demonstrated in animal test methods as well as in clinic, for example in accordance with the methods hereinafter described.

A Binding affinity of PKC inhibitors to individual human PKC may be determined in an Allogeneic Mixed Lymphocyte Reaction (MLR) assay. MLR assay can be done according to known methods, e.g. mouse of human MLR assay, e.g. as disclosed in EP1337527A1, the content regarding the MLR assay being incorporated herein by reference.

B In vivo

Efficacy in the described ocular disorders might be established for example in the following animal models:

1) Genetic animal models for retinal degeneration, e.g. rd mouse (as described in Li et al., Invest. Ophthalmol. Vis. Sci. 2001; 42: 2981-2989), Rpe65-deficient mouse (Van Hooser et al., PNAS 2000; 97: 8623-8628), RCS rat (Faktorovich et al., Nature 1990; 347:83-86), rds mouse (Ali et al., Nature Genetics 2000, 25: 306-310), rcd1 dog (Suber et al., PNAS 1993; 90: 3968-3972)

2) Experimental retinal degeneration induced by
light exposure in mice (as described in Wenzel et al., Invest. Ophthalmol. Vis. Sci. 2001; 42: 1653-1659) or rats (Faktorovich et al., J. Neurosci: 1992; 12: 3554-3567)
administration of N-methyl-N-nitrosourea (Kiuchi et al., Exp. Eye Res. 2002; 74: 383-392) or sodium iodate (Sorsby & Harding, Vision Res. 1962; 2: 139-148).

3) Experimental model for the injury of the optic nerve (ON)
by ON crush in mice (Levkovitch-Verbin et al., Invest. Ophthalmol. Vis. Sci. 2000; 41: 4169-4174) and rats (Yoles and Schwartz, Exp. Neurol. 1998; 153:1-7)
by ON transection in rats (as described in Martin et al., Invest. Ophthalmol. Vis. Sci. 2002; 43: 2236-2243, Solomon et al. J. Neurosci. Methods 1996; 70:21-25)
by experimental transient (acute) retinal ischemia in rats after ophthalmic vessel ligature (as described in Lafuente et al., Invest. Ophthalmol. Vis. Sci. 2001; 42:2074-2084) or cannulation of the anterior chamber (Buchi et al., Ophthalmologica 1991; 203:138-147)
by intraocular endothelin-1 injection in rats (Stokely at al., Invest. Ophthalmol. Vis. Sci. 2002; 43: 3223-3230) or rabbits (Takei et al., Graefes Arch. Clin. Exp. Ophthalmol 1993; 231:476-481)

4) Laser-Induced Choroidal neovascularization (CNV)

A laser is directed through the lens of the eye to the retina, rupturing Bruchs membrane and eliciting a neovascular response from the choroid through the burn hole into the inner retina. The compound is administered just prior or immediately following lasering, and the neovascularization is allowed to progress for 7-14 days. At the end of this time the animals are euthanized and the area of neovascular membrane is measured. (See Kwak et al, (2000) VEGF is Major Stimulator in Model of Choroidal Neovascularization. Investigative Ophthalmology and Vision Science. 41(10); 3158-64.)

5) Experimental autoimmune uveioretinitis

Uveitis is elicited in rats by immunizing with bovine retinal antigen. Due to an effector T-lymphocyte response, the retina is irreversibly damaged as measured by clinical severity of ocular inflammation and/or histology. Disease phenotype develops between day 10-12 after immunization and reaches maximal values 1-2 days later. Compound is administered from the time of immunization and allowed to progress for 14 days. Rats are clinically assessed starting day 10 to day 14. At the end of this time, rats are euthanized and eyes are characterized histologically. (See Wacker WB et al, (1977) Experimental allergic uveitis. Isolation, characterization, and localization of a soluble uveitopathogenic antigen from bovine retina. J Immunol. 119:1949-1958)

C Clinical Trial

Suitable clinical studies are, e.g., randomized, double-masked, placebo-controlled clinical studies in patients with age-related macular degeneration, diabetic retinopathy, diabetic macular edema (DME) or uveitis. Such studies may also be suitable to compare the effects of a monotherapy using compounds of formula I or IIa as active ingredient or a combination of such compounds with a second drug substance.

For example, 200 patients with DME diagnosis receive the test compound, e.g a compound of formula I or IIa, or a pharmaceutically acceptable salt thereof, e.g. Compound A, B, C, D or E, at a daily dosage of e.g. 50, 200 or 400 mg or placebo administered p.o BID. Percent change in macular edema is assessed at month 3 and compared to baseline. Thus, for example, macular thickness is measured with optical coherence tomography (OCT) and edema thickness calculated from the average thickness in the central subfield of the six-radial scan map with a correction of 175 microns, representing the normal macular thickness. A beneficial effect is observed with the test compounds.

In yet a further aspect, 200 patient with AMD receive the test compound, e.g a compound of formula I or IIa, or a pharmaceutically acceptable salt thereof, e.g. Compound A, B, C, D or E, at a daily dosage of e.g. 50, 200 or 400 mg or placebo administered p.o BID. Percent change in macular edema is assessed at month 1 and compared to baseline. Thus, for example, macular thickness is measured with optical coherence tomography (OCT) and edema thickness calculated from the average thickness in the central subfield of the six-radial scan map with a correction of 175 microns, representing the normal macular thickness. A beneficial effect is observed with the test compounds.

In yet a further aspect, 200 patients with uveitis receive the test compound, e.g a compound of formula I or IIa, or a pharmaceutically acceptable salt thereof, e.g. Compound A, B, C, D or E, at a daily dosage of e.g. 50, 200 or 400 mg or placebo administered p.o BID. Percent change in ocular inflammation is assessed at month 2 and compared to baseline. Thus, for example, ocular inflammation is assessed by the designated clinician and severity of vitreous haze is determined according to an established scale. A beneficial effect is observed with the test compounds.

According to the invention, the compounds of formula (I) and (IIa) may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula (I) and (IIa) in free form or in pharmaceutically acceptable salt form in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

Preferably, the compound are administered topically, e.g. to the skin. A even more preferred for form of topical administration is to the eye.

Daily dosages required in practicing the method of the present invention will vary depending upon, for example, the compound used, the host, the mode of administration, the severity of the condition to be treated. An indicated daily dosage for oral administration in the larger mammal, e.g. humans, is in the range from about 0.5 mg to about 2000 mg active ingredient, e.g. Compound A, B or C, conveniently administered, for example, in divided doses up to four times a day or in retard form.

The required dosage will of course vary depending on the mode of administration, the particular condition to be treated and the effect desired. In general, satisfactory results are indicated to be obtained systemically at daily dosages of from about 0.1 to about 100 mg/kg body weight. An indicated daily dosage in the larger mammal, e.g. humans, is in the range from about 0.5 mg to about 2000 mg, conveniently administered, for example, in divided doses up to four times a day or in retard form.

The PKC inhibitors, e.g. compounds of formula (I) or (IIa), may be administered as the sole active ingredient or together with other agents used for treating or preventing ocular diseases and disorders, in particular ocular diseases and disorders involving inflammation and/or neovascularization. For example, they may be used in combination with anti-angiostatic drug, or staurosporine derivative or a salt thereof and/or S1P receptor agonist, or a salt thereof.

Anti-angiostatic drug may include, but is not limited to, Visudyne® (verteporfine, described in U.S. Pat. No. 5,095,030 and EP 3520076), Macugen® (pegaptanib sodium), Retaane (anecortave acetate), EVIZON™ (Squalamine Lactate), VEGF Inhibitor (vascular endothelial growth factor), such as e.g. Lucentis® (ranibizumab) or Vatalanib.

Staurosporine derivative or a salt thereof are e.g. described in EP 113107361, U.S. Pat. No. 5,093,330, the description of the staurosporine derivatives in these patents being herein incorporated by reference.

According to the present invention preferred staurosporine derivatives include: N-(3-carboxypropionyl)-staurosporine, N-benzoyl-staurosporine, N-trifluoracetyl-staurosporine, N-methylaminothiocarbonyl-staurosporine, N-phenylcarbamoyl-staurosporine, N-(3-nitrobenzoyl)-staurosporine, N-(3-fluorobenzoyl)-staurosporine, N-tert-butoxycarbonyl-staurosporine, N-(4-carboxybenzoyl)-staurosporine, N-(3,5-dinitrobenzoyl)-staurosporine, N-alanyl-staurosporine, N-ethyl-staurosporine, N-carboxymethyl-staurosporine, N-[(tert.-butoxycarbonylamino)-acetyl]-staurosporine, N-(2-aminoacetyl)-staurosporine, and pharmaceutically acceptable salts thereof.

S1P receptor agonists are compounds which signal as agonists at one or more sphingosine-1 phosphate receptors, e.g. S1P1 to S1P5. Agonist binding to a S1P receptor may e.g. result in dissociation of intracellular heterotrimeric G-proteins into Gα-GTP and Gβγ-GTP, and/or increased phosphorylation of the agonist-occupied receptor and activation of downstream signaling pathways/kinases.

S1 P receptor agonists are typically sphingosine analogues, such as 2-substituted 2-amino-propane-1,3-diol or 2-amino-propanol derivatives, e. g.
2-amino-2-tetradecyl-1,3-propanediol, particularly preferred is FTY720, i.e. 2-amino-2-[2-(4-octylphenyl)ethyl]propane-1,3-diol in free form or in a pharmaceutically acceptable salt form, e.g. the hydrochloride, as shown:

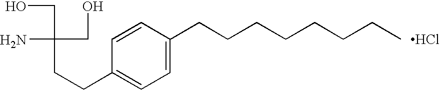

2-amino-2-{2-[4-(1-oxo-5-phenylpentyl)phenyl]ethyl}propane-1,3-diol, in free form or in pharmaceutically acceptable salt form, e.g. the hydrochloride;
2-amino-4-(4-heptyloxyphenyl)-2-methyl-butanol, in free form or in pharmaceutically acceptable salt form, e.g. the hydrochloride, more particularly the R-enantiomer;
FTY720-phosphate;
phosphoric acid mono-[(R)-2-amino-2-methyl-4-(4-pentyloxy-phenyl)-butyl]ester;
2-amino-2-[4-(3-benzyloxyphenylthio)-2-chlorophenyl]ethyl-propane-1,3-diol and its corresponding phosphate derivative, phosphoric acid mono-2-amino-2-[4-(3-benzyloxyphenylthio)-2-chlorophenyl]ethyl-propyl]ester.
(2R)-2-amino-4-[3-(4-cyclohexyloxybutyl)-benzo[b]thien-6-yl]-2-methylbutan-1-ol,
2-amino-4-[4-(3-benzyloxyphenylthio)-2-chlorophenyl]-2-methylbutane-1-ol; the corresponding phosphoric acid mono-2-amino-4-[4-(3-benzyloxyphenylthio)-2-chlorophenyl]-2-methylbutyl]ester; 2-amino-4-[4-(3-benzyloxyphenylthio)-2-chlorophenyl]-2-ethylbutane-1-ol; and the corresponding phosphoric acid mono-2-amino-4-[4-(3-benzyloxyphenylthio)-2-chlorophenyl]-2-ethylbutyl]ester;
1-{4-[1-(4-cyclohexyl-3-trifluoromethyl-benzyloxyimino)-ethyl]-2-ethyl-benzyl)-azetidine-3-carboxylic acid, or a prodrug thereof.

Where the PKC inhibitors are administered in conjunction with other drugs, dosages of the co-administered compound will of course vary depending on the type of co-drug employed, on the specific drug employed, on the condition to be treated, and so forth. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The PKC inhibitors, e.g. compounds of formula (I) or (IIa), preferably Compound A, B, C, D or E, may be administered in photodynamic therapy (PDT), i.e. in combination with light administration and administration of light sensitive agents in an oxygen-rich environment.

In accordance with the foregoing the present invention provides in a yet further aspect:
6. A pharmaceutical combination comprising a) a first agent which is a PKC inhibitor, e.g.

a compound of formula (I) or (IIa), preferably Compound A, B, C, D or E, or a pharmaceutically acceptable salt thereof, and b) a second drug agent as defined above.
7. A method as defined above comprising co-administration, e.g. concomitantly or in sequence, of a therapeutically effective amount of a PKC inhibitor, e.g. a compound of formula (I) or (IIa), preferably Compound A, B, C, D or E, or a pharmaceutically acceptable salt thereof, and a second drug substance, e.g. as indicated above.
8. A method for treating, preventing, or delaying their progression, said method comprising administering to an affected individual a therapeutically effective amount of a pharmaceutical combination comprising a) a first agent which is a PKC inhibitor, e.g. a compound of formula (I) or (IIa), preferably Compound A, B, C, D or E, or a pharmaceutically acceptable salt thereof, and b) a staurosporine derivative or a salt thereof and/or a S1P receptor agonist, or a salt thereof; and optionally a pharmaceutically acceptable carrier.
9. Use of a combination as defined above for the preparation of a medicament for treating, preventing or delaying ophthalmic diseases and disorders in particular involving inflammatory and/or neovascular events, or delaying their progression, in particular age-related macular degeneration, retinal disease or disorder or diabetic ocular diseases or disorders as hereinabove defined.

The administration of a pharmaceutical combination of the invention results in a beneficial effect, especially a synergistic effect. For example combined treatment can result in surprising prolongation of efficacy, less side-effects, lower doses of the individual drugs or improved quality of life, compared to a monotherapy. A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

With respect to the combinations according to the present invention as described hereinbefore and hereinafter they may be used for simultaneous use or sequential use in any order, e.g. for separate use or as a fixed combination.

The combinations according to the present invention comprises a “kit of parts” in the sense that both agents a and b can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration. A physician, clinician or veterinarian of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Preferred compounds of the invention are 3-(1.H.-indol-3-yl)-4-[2-(4-methyl-piperazin-1-yl)-quinazolin-4-yl]-pyrrole-2,5-dione, 3-(1.H.-indol-3-yl)-4-[2-(piperazin-1-yl)-quinazolin-4-yl]-pyrrole-2,5-dione, 3-[3-(4,7-Diaza-spiro[2.5]oct-7-yl)-isoquinolin-1-yl]-4-(7-methyl-1H-indol-3-yl)-pyrrole-2,5-dione, in free form or in a pharmaceutically acceptable salt form, e.g. the acetate salt of 3-(1.H.-indol-3-yl)-4-[2-(4-methyl-piperazin-1-yl)-quinazolin-4-yl]-pyrrole-2,5-dione, or 3-[3-(4,7-Diaza-spiro{2.5}oct-7-yl)-isoquinolin-1-yl]-4-(7-methyl-1H-indol-3-yl)-pyrrole-2,5-dione.

1. A method of treating or preventing age-related macular degeneration, retinal disease or disorder or diabetic ocular diseases or disorders, comprising administration to a subject in need of such treatment of an effective amount of a PKC inhibitor of formula (I) 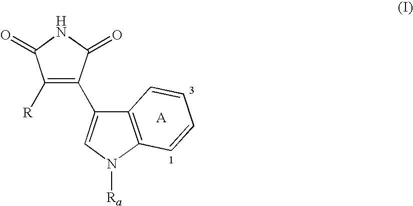
wherein
Rₐ is H; C₁₋₄alkyl; or C₁₋₄alkyl substituted by OH, NH₂, NHC₁₋₄alkyl or N(di-C₁₋₄alkyl)₂; and
R is a radical of formula (a) or (b) 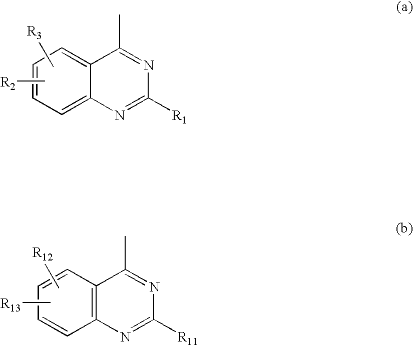
wherein
each of R₁ and R₁₁ is a heterocyclic residue; NR₄R₅ wherein R₄ and R₅ form together with the nitrogen atom to which they are bound a heterocyclic residue;
each of R₂, R₃, R₁₂ and R₁₃, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN; and
ring A is optionally substituted,
or a pharmaceutically acceptable salt thereof,
or of formula (IIa) 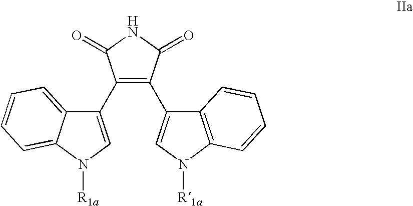
wherein
R₁ₐ is 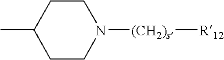
wherein either s′ is 0 and R′₁₂ is hydrogen or C₁₋₄alkyl; or s′ is 1 and R′₁₂ is pyridyl, preferably 2-pyridyl, and
R′₁ₐ is hydrogen or C₁₋₄alkyl,
or a pharmaceutically acceptable salt thereof

2. Method according to claim 1 wherein the diabetic ocular disease or disorder is diabetic retinopathy or diabetic macular edema.

3. Method according to claim 1 wherein the PKC inhibitor is selected from 3-(1.H.-indol-3-yl)-4-[2-(4-methyl-piperazin-1-yl)-quinazolin-4-yl]-pyrrole-2,5-dione, 3-(1.H.-indol-3-yl)-4-[2-(piperazin-1-yl)-quinazolin-4-yl]-pyrrole-2,5-dione, 3-[3-(4,7-Diaza-spiro[2.5]oct-7-yl)-isoquinolin-1-yl]-4-(7-methyl-1H-indol-3-yl)-pyrrole-2,5-dione, and a pharmaceutically acceptable salt thereof.

4. Method according to claim 3 wherein the PKC inhibitor is an acetate salt of 3-(1.H.-indol-3-yl)-4-[2-(4-methyl-piperazin-1-yl)-quinazolin-4-yl]-pyrrole-2,5-dione, or 3-[3-(4,7-Diaza-spiro[2.5]oct-7-yl)-isoquinolin-1-yl]-4-(7-methyl-1H-indol-3-yl)-pyrrole-2,5-dione.

5. Method according to claim 1 wherein the PKC inhibitor is selected from 3-(1-methyl-1H-indol-3-yl)-4-[1-{(1-pyridin-2-ylmethyl)-piperidin-4-yl}-1H-indol-3-yl]-pyrrole-2,5-dione, 3-(1-methyl-1H-indol-3-yl)-4-[1-(piperidin-4-yl)-1H-indol-3-yl]-pyrrole-2,5-dione and a pharmaceutically acceptable salt thereof.

6. Method according to claim 1 wherein the PKC inhibitor is administered together with a second drug selected from an anti-angiostatic drug, a staurosporine derivative, a S1P receptor agonist, and a salt thereof.

7. A pharmaceutical composition for use in a method according to claim 1 comprising a PKC inhibitor of formula (I) or (IIa), as defined in claim 1 or a pharmaceutically acceptable salt thereof, together with one or more pharmaceutically acceptable diluents or carriers.

8. A pharmaceutical combination comprising a) a compound of formula (I) or (IIa), as defined in claim 1, and b) a co-agent which is selected from an anti-angiostatic drug, a staurosporine derivative, a S1P receptor agonist, and a salt thereof.

9. A combination according to claim 8 wherein the a compound of formula (I) is selected from 3-(1.H.-indol-3-yl)-4-[2-(4-methyl-piperazin-1-yl)-quinazolin-4-yl]-pyrrole-2,5-dione, 3-(1.H.-indol-3-yl)-4-[2-(piperazin-1-yl)-quinazolin-4-yl]-pyrrole-2,5-dione, 3-[3-(4,7-Diaza-spiro[2.5]oct-7-yl)-isoquinolin-1-yl]-4-(7-methyl-1H-indol-3-yl)-pyrrole-2,5-dione, and a pharmaceutically acceptable salt thereof.

10. A combination according to claim 9 wherein the a compound of formula (I) is an acetate salt of 3-(1.H.-indol-3-yl)-4-[2-(4-methyl-piperazin-1-yl)-quinazolin-4-yl]-pyrrole-2,5-dione, or 3-[3-(4,7-Diaza-spiro[2.5)oct-7-yl)-isoquinolin-1-yl]-4-(7-methyl-1H-indol-3-yl)-pyrrole-2,5-dione.
